(19) United States
(12) Patent Application Publication
(10) Pub. No.: US 2005/0261755 A1
(43) Pub. Date: Nov. 24, 2005

(54) Conformable heat transfer pack

(76) Inventors: John Edward Bacino, Landenberg, PA (US); Lois J. Mabon, Landenberg, PA (US)

Correspondence Address: GORE ENTERPRISE HOLDINGS, INC., 551 PAPER MILL ROAD, P. O. BOX 9206, NEWARK, DE, 19714-9206, US

(21) Appl. No.: 10/852,944

(22) Filed: May 24, 2004

(51) Int. Cl. A61F 7/00

(52) U.S. Cl. 607/114

(57) ABSTRACT

An improved heat transfer pack incorporating a highly conformable waterproof, moisture vapor permeable outer layer is provided. The heat transfer pack may enclose a heat transfer material within a highly conformable moisture vapor impermeable inner pouch.

### BACKGROUND

The present invention relates to heat transfer packs, such as hot or cold packs, usable for therapeutic purposes.

Therapeutic applications for heat transfer devices are well known. Cold packs are excellent for reducing pain and inflammation from acute injuries. The numbing effect of a cold pack provides an analgesic effect that can extend below the skin. Pain associated with bruises, sprains and strains can be relieved by applying a cold pack. Heat too is frequently applied to the body to relieve pain, including joint pain associated with arthritis. Hot packs are also useful to increase local circulation and to loosen stiff joints. Applying heat prior to exercise may reduce the chance of injury.

Known cold packs include traditional ice packs as well as packs containing water and alcohol mixtures in liquid or gel form. Typically, such mixtures are enclosed in an impermeable container or pouch to prevent evaporation of the volatile components.

It is desirable that a heat transfer pack be highly conformable to the body. Conformity is necessary for both comfort and for maximum heat transfer efficiency. Wherever the surface of the cold pack separates from the skin, an insulating pocket of air forms which inhibits heat transfer. Known cold packs are stiff and do not conform well to the body.

Traditional ice packs have large, relatively warm air spaces between ice cubes, which can only be partially mitigated by inconvenient crushing of the cubes. Traditional gel-filled packs also fail to conform well because the impermeable plastic pouches which contain the gel are stiff at low temperatures.

It is also desirable to prevent condensation and frost from forming on the surface of an ice pack upon removal from cold storage, such as in a freezer. Such condensation may contribute to an uncomfortable condition known as ice burn or even frostbite. Moreover, when such condensation melts, dripping water creates discomfort and inconvenience.

To prevent ice burn and frostbite, a heat transfer pack must also have adequate thermal resistance. Thermal resistance is a measure of the resistance to the flow of heat. With regard to heat transfer packs, thermal resistance reflects the ability of a pack containing a hot or cold heat transfer material to be applied to the skin without discomfort.

Traditional ice packs or gel packs are often wrapped in fabrics, such as cotton towels or synthetic materials to absorb condensation and increase thermal resistance. However, these woven or non-woven fabrics may eventually become saturated. As the fabric becomes saturated, its thermal resistance decreases and the heat transfer pack becomes uncomfortable. Even before saturation, known fabric covers reduce the ability of the cold pack to conform to the body, which interferes with heat transfer efficiency.

Information relevant to attempts to address these problems can be found in U.S. Pat. Nos. 4,910,978 and 4,688,572. However, each of these references suffers from one or more of the disadvantages described above.

Accordingly, there is a need for a highly conformable heat transfer pack with adequate thermal resistance that reduces surface condensation and frosting of the surface.

### SUMMARY

In one aspect, the present invention is a heat transfer pack comprising a heat transfer material and a pouch surrounding the heat transfer material, the pouch comprising a moisture vapor impermeable inner layer and a waterproof, moisture vapor permeable outer layer.

In another aspect, the invention provides an article for containing heat transfer material, the article comprising a nonlaminated composite pouch, the pouch comprising a waterproof, moisture vapor permeable outer layer substantially surrounding a moisture vapor impermeable inner layer.

In yet another aspect, the present invention is an article for containing heat transfer material comprising a waterproof, moisture vapor permeable pouch having an inner surface and an outer surface, the pouch having an opening therein of sufficient size to permit insertion and removal of heat transfer material.

In a further aspect, the invention provides a pouch comprising a conformable insulating layer, such that the quotient of the thermal resistance divided by the fabric hand of the pouch is greater than about 0.05, and the pouch having a fabric hand value of less than about 100 g, more preferably, less than about 90 g, and most preferably less than about 75 g, 50 g and 30 g.

In yet another aspect, the invention provides a heat transfer pack having an outer layer comprising ePTFE or porous polyethylene.

In yet another aspect, the invention provides a heat transfer pack having an outer layer which is a laminate comprising a porous membrane and at least one textile layer. In this aspect, the textile layer may be a polyester fabric.

In still another aspect, the invention provides for a heat transfer pack having an outer layer comprising a porous membrane and a filler. In this aspect, the filler may be selected from the group of: pigments, colorants, scents, medicants, anti-microbials, antibiotics, antibacterial agents, antifungals, dentifrice, remineralizing agents, immunological agents, anti-inflammatory agents, hemostatic agents, analgesics and mixtures thereof.

In another aspect, the invention includes a heat transfer pack in which the outer layer is oleophobic.

In another aspect, the invention includes a heat transfer pack in which the outer layer is hydrophobic.

### DESCRIPTION OF THE DRAWINGS

The operation of the present invention should become apparent from the following description when considered in conjunction with the accompanying drawings, in which:

FIG. 1 is a three quarter isometric view of the heat transfer pack, with its core shown in partial cutaway.

FIG. 2 is a top view of the heat transfer pack in accordance with one embodiment.

FIG. 3 is a cross sectional view of the heat transfer pack along line 3-3 of FIG. 2.

FIG. 4 is a perspective view of another embodiment of the present invention.

FIG. 5 is a perspective view of another embodiment of the present invention.

FIG. 6 is a perspective view of another embodiment of the present invention showing securing straps with hook and loop style fasteners.

FIG. 7 is a top view of another aspect showing a shaped conformable cold pack for use as a facial pack.

FIG. 8 is a top view of another aspect showing an elongated, conformable cold pack for use around the shoulders and neck of the user.

### DETAILED DESCRIPTION

The present invention is directed to a heat transfer pack for containing a heat transfer material. The heat transfer pack is fabricated from improved materials and comprises waterproof, moisture vapor permeable components.

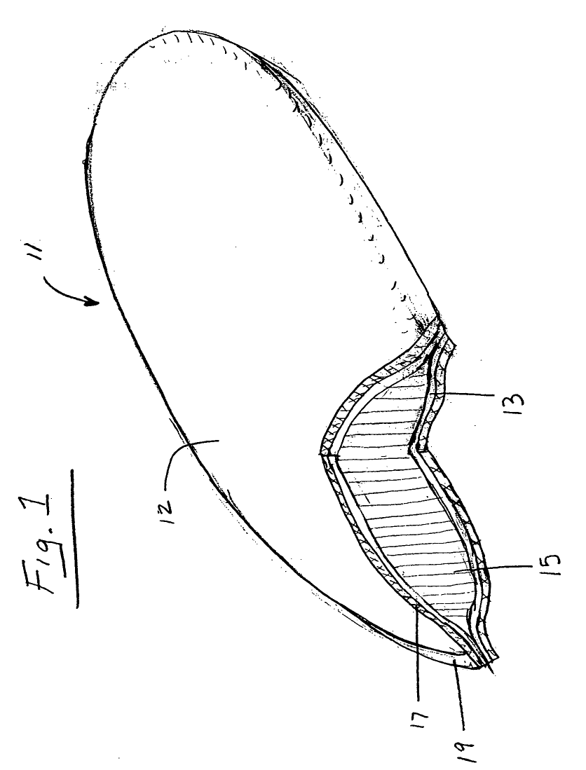

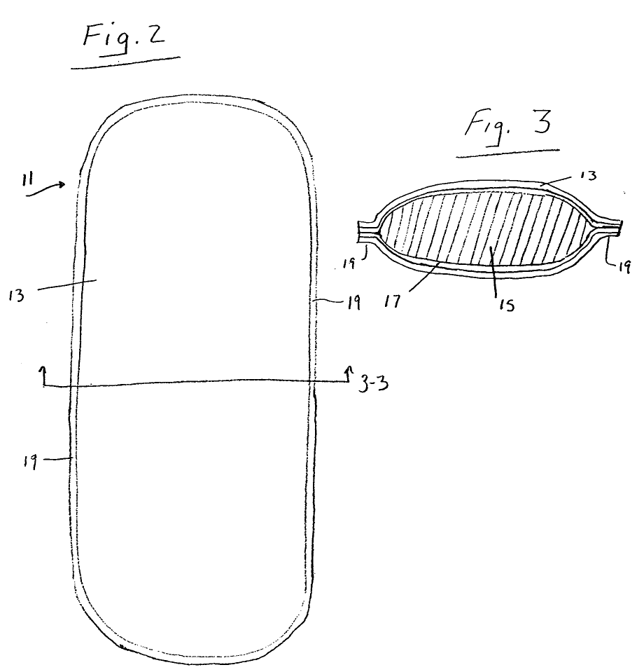

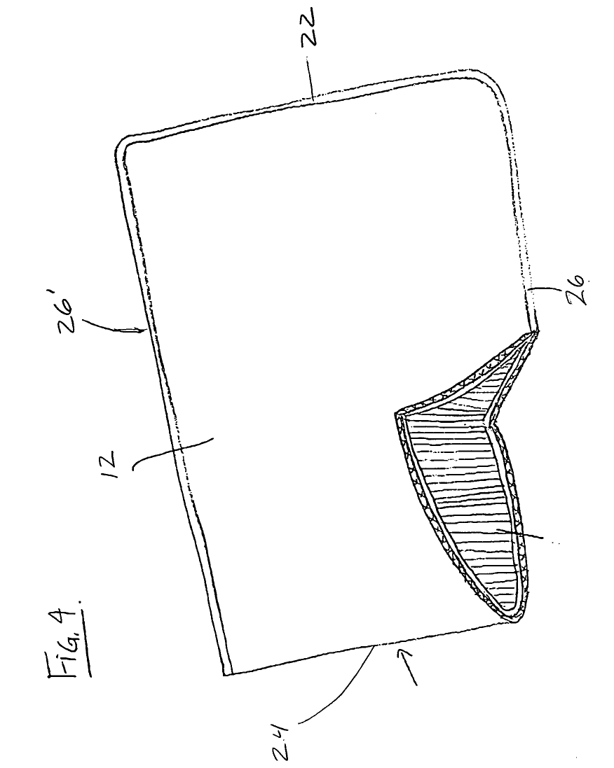

With reference to FIGS. 1-3, the heat transfer pack 11 comprises a heat transfer material 15, which may be a liquid, a gel or a solid. In one embodiment, the heat transfer material chosen is a gel or liquid and is contained within a multilayer, non-laminated pouch. The inner layer 13 is moisture and moisture vapor impermeable to prevent evaporation of volatile components. The outer layer 17 is moisture impermeable and moisture vapor permeable. The inner layer and outer layer are bonded together at their periphery 19 to form the pouch. In another embodiment, the heat transfer material is a solid, such as a clay. In this embodiment, the heat transfer material may be contained within a single-layer pouch constructed of waterproof, moisture vapor permeable material.

The heat transfer material may be any known suitable material having the desirable properties of thermal capacity and conformability. Those of skill in the art will be aware of a variety of suitable liquids and gels. Known solid heat transfer materials, such as rice, peas, clays or crushed ice, may also be used, provided that the material is sufficiently fine to permit the heat transfer pack to readily conform to the body.

Preferably, the heat transfer material will be a gel. Refrigeratable gels may comprise mixtures of water, non-toxic freezing point. suppressants, such as propylene glycol, and thickening agents. Optionally, ingredients known to inhibit bacterial growth may be added. For therapeutic applications, the gel should be conformable to a low temperature of about 10 degrees F.

The inner layer of the multi-layer heat transfer pack may comprise a moisture vapor impermeable pouch to contain the heat transfer material and prevent evaporation of any volatile components. This inner layer may be of any conformable, moisture vapor impermeable material. As used herein, “moisture vapor impermeable” means sufficiently vapor impermeable to prevent evaporation of volatile components of the heat transfer material used. Preferably, the inner layer is made from a thermoplastic to facilitate construction of the heat transfer pack. Polyethylene, and many melt processable fluoropolymers, such as FEP and EFEP, for example, are conformable at low temperatures and may be bonded easily by heat sealing and other methods to form the moisture vapor impermeable pouch. FEP, PFA, THV, PVDH, PU and PE may also be used.

The thickness of the inner layer is not critical; however, overly thick materials may not provide the desired conformability and excessively thin materials may not be durable. When the moisture vapor impermeable pouch is EFEP, a pouch thickness of greater than about 0.5 mils to less than about 3 mils is acceptable. Preferably, the EFEP pouch thickness will be about 1 mil.

The outer layer of the multi-layer heat transfer pack comprises a waterproof, moisture vapor permeable cover for the moisture vapor impermeable pouch. The outer layer may be of any suitable porous material having the properties of vapor permeability, liquid impermeability and good conformability at applicable temperatures. The outer layer may be a membrane or laminate comprising a membrane and one or more textile layers to enhance comfort or durability.

The cover is preferably hydrophobic. A film of porous, ePTFE, which has been heated above its crystalline melt point after expansion, has been found to be an ideal hydrophobic cover material. These hydrophobic films are highly porous, yet the pores are very small in size which leads to high water entry pressures. U.S. Pat. No. 3,953,566 describes the preparation of the desirable microporous, ePTFE hydrophobic films.

Other hydrophobic materials for use in the cover include highly crystalline films of ePTFE, which have not been heated above their crystalline melt point, and films of other microporous hydrophobic polymers such as polyethylene, which are waterproof and possess the desired moisture vapor transmission characteristics.

The cover may optionally be treated such that it is oleophobic to reduce staining due to skin oils and prolong the life of the heat transfer pack.

The cover may optionally include one or more fillers, also referred to as additives. For example, where the cover is an ePTFE membrane, which has a structure of nodes interconnected by fibrils defining interconnected passages and pathways, additives may be included in the matrix of the ePTFE itself. Desirable additives may include colorants, pigments, scents, medicants, anti-microbials, antibiotics, antibacterial agents, antifungals, dentifrice, remineralizing agents, immunological agents, anti-inflammatory agents, hemostatic agents, analgesics and mixtures thereof.

The cover may also be a laminate comprising a membrane, such as ePTFE, combined with one or more textile layers. These layers may include fabrics, such as woven or non woven textiles or knits and may be treated, for example for moisture or and or oil repellency.

The thickness of the cover should be selected to optimize the properties of good conformability with acceptable durability. Where the cover is ePTFE, it has been found that an ePTFE thickness of less than about 10 mil to greater than about 0.5 mil is preferred. Most preferably, the thickness is less than about 5 mil to greater than about 0.8 mil.

The moisture vapor transmission rate through the cover should be above 1000 g/m² day and is preferably above 2000 g/m² day.

Numerous techniques may be used to fabricate the heat transfer pack. The inner impermeable pouch and the cover may be formed independently, such that the two layers are not attached in any way. Optionally, the cover may be removable from the pouch such that other impermeable pouches containing different or the same heat transfer material may be exchanged. In this way, the heat transfer material can be conveniently replaced.

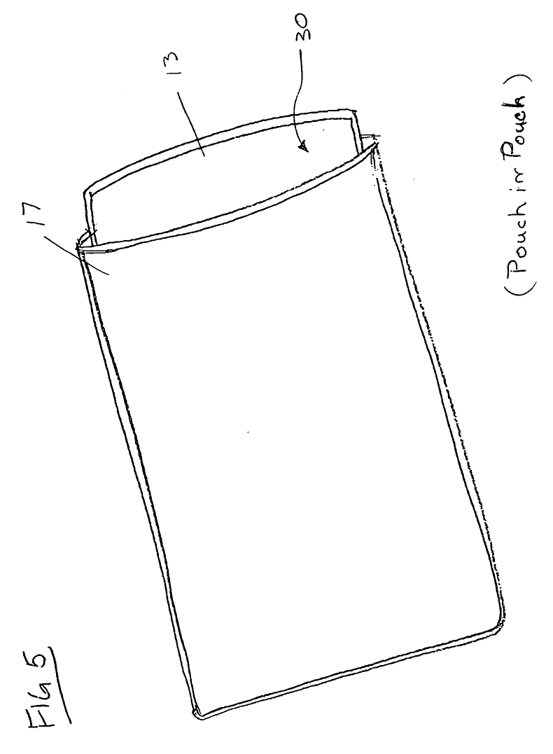

In a preferred method of construction, the inner impermeable pouch and the cover are formed together and bonded at their periphery to form the multi-layer heat transfer pack. A single strip of moisture vapor impermeable material may be positioned in an overlapping relation to a strip of waterproof, moisture vapor permeable material of like length and width. Both materials are then folded together along an axis approximately at their center. As shown in FIG. 4, the resulting edge 22 opposite the fold 24 and the ends of the strip 26,26′ can then be sealed, for example by heat sealing, to form the multi-layer heat transfer pack. A small gap may be temporarily left in the bonding for filling the inner impermeable pouch with the heat transfer material. After adding the heat transfer material, the pouch is permanently sealed.

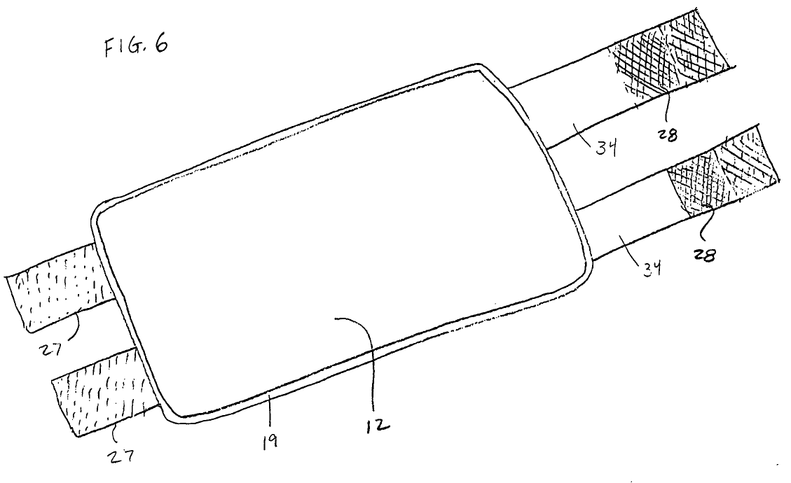

In another aspect, shown in FIG. 5, the inner impermeable pouch 30 is removable from the cover. Optionally a closure such as a zipper or hook and loop fasteners and the like (not shown) may be incorporated into the cover to retain the impermeable pouch.

In still another aspect, the heat transfer pack is constructed of a moisture vapor impermeable pouch with a waterproof, moisture vapor permeable patient contacting surface bonded to the pouch. A sheet of moisture vapor impermeable material is folded about a central axis as described above. The folded sheet is placed on top of a sheet of waterproof, moisture vapor permeable material. Both materials are then heat sealed at the periphery of the folded impermeable material to form a non-laminated pouch. If necessary, excess waterproof, moisture vapor permeable material may be trimmed away. In use, the waterproof, moisture vapor permeable layer forms a highly conformable patient contacting surface which minimizes skin contact with condensation at the surface of the heat transfer pack.

Those of skill in the art will be aware of several other techniques for bonding the inner and outer materials at the periphery. Heat sealing is effective in many applications. However, RF and ultrasonic welding are also useful techniques. Adhesives and adhesive strips may be used if the materials chosen are compatible with the adhesive.

In another aspect, the heat transfer pack may be constructed of a single layer of waterproof, moisture vapor permeable material. If the selected heat transfer material is, for example, a fine grain solid or is nonvolatile, then evaporation is not a concern and the moisture vapor impermeable inner pouch may not be required. Single-layer construction may also be advantageous if the heat transfer material is replaceable through a reclosable opening. For example, the single layer pack may have a reclosable, waterproof zipper allowing crushed ice to be placed within. Although some moisture vapor may escape through the porous, single layer heat transfer pack, it is readily and inexpensively replaced by adding additional crushed ice.

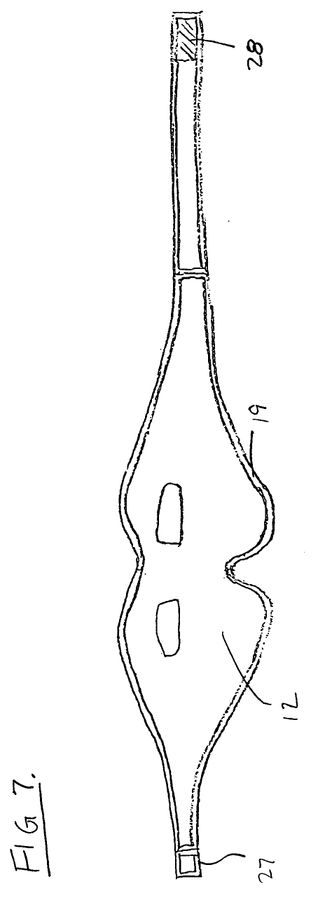

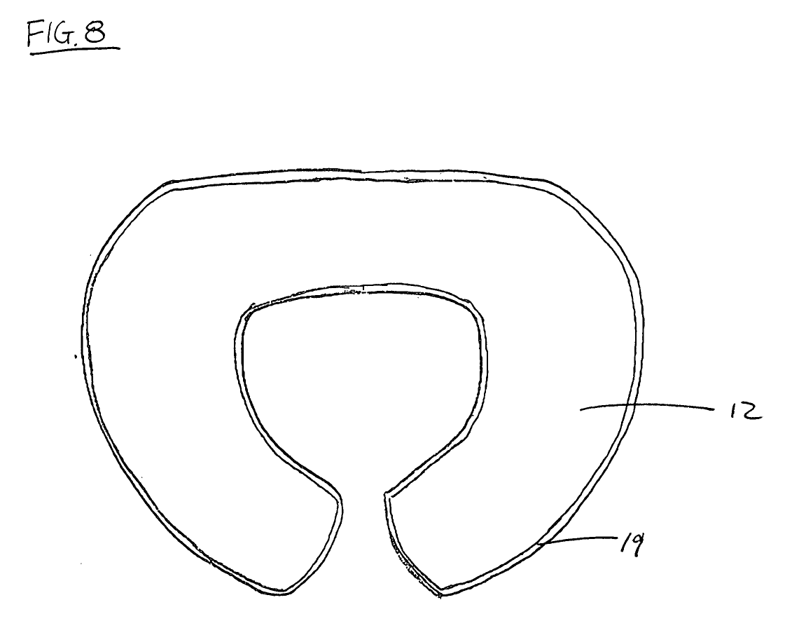

FIG. 6 shows an alternative embodiment of the invention wherein the cover includes flexible elastic straps 34 attached to the edges of the pack. The straps are used to hold the heat transfer pack to the affected area of the patient. Preferably, the straps incorporate hook and loop fasteners (27, 28) and other means of conveniently securing the elastic straps.

The novel heat transfer packs described herein are highly conformable and are resistant to forming ice or condensation on their outer surface. Excellent conformability allows for improved comfort and heat transfer efficiency. Frost and condensation resistance effectively prevents painful ice burn and frostbite and the inconvenience of dripping condensate from the surface.

### DEFINITIONS

“Conformability” means the aggregation of such properties as thinness, flexibility and softness which permit an article to cover an irregular substrate, such as a body part, without excessive stiffness, wrinkles or folding. One measure of conformability is fabric hand, or “hand.” As applied to heat transfer packs, improved hand can improve both thermal transfer and comfort by eliminating folds, creases and resulting air gaps between the user and the heat transfer material.

“Hydrophobic,” as used herein, means that water will not spread on the material and wick into its porous structure. A drop of water placed on the surface of a highly hydrophobic material will remain in the form of a nearly spherical bead with an advancing water contact angle greater than 90 degrees.

### Test Methods

Waterproofness

Laminates are tested for waterproofness by using a modified Suter test apparatus, which is a low water entry pressure challenge. Water is forced against a sample area of about 4¼ inch diameter sealed by two rubber gaskets in a clamped arrangement. The sample is open to atmospheric conditions and is visible to the operator. The water pressure on the sample is increased to about 1 psi by a pump connected to a water reservoir, as indicated by an appropriate gauge and regulated by an in-line valve. The test sample is at an angle and the water is recirculated to assure water contact and not air against the sample's lower surface. The upper surface of the sample is visually observed for a period of 3 minutes for the appearance of any water which would be forced through the sample. Liquid water seen on the surface is interpreted as a leak. A passing (waterproof) grade is given for no liquid water visible within 3 minutes. Materials passing this test are “waterproof” as used herein.

Hand

Hand, or stiffness of a material, can be measured using a force resistance device such as a Handle-O-Meter, Model No. 211-305, manufactured by the Thwing-Albert Instrument Company, Philadelphia. Pa. This device measures the flexibility of sheet materials by forcing the test sample through an adjustable slot opening on the instrument platform. The device utilizes a penetrator blade to engage the sample and force it into the slot. The resistance encountered by the penetrator blade as it moves into the slot is measured using a 1000 gram beam and is displayed to the operator.

The materials for each sample set are cut into 4 inch squares and layered to include the outer and inner heat transfer pouch material (where appropriate). The samples are conditioned in a container at a temperature of 5 degrees C. and a relative humidity of 65% for a minimum of 15 minutes prior to initiating the tests. The 4″ square samples are removed from the environmental container and immediately placed uniformly across the slot opening which has been adjusted to a ¼″ gap. The samples are oriented such that 1″ of the sample material extends to the left of the slot opening for the first test. As the test is conducted, the penetrator blade automatically pivots on a cam, engages the sample and forces it into the slot. The sample's resistance to bending into the slot is measured by the 1000 gram beam and is displayed. The result is reported as the peak force required to bend and push the sample through the slot. This test is performed two times as described above per material construction, with the sample being returned to the conditioning container for a minimum of 15 minutes prior to each test. For the second test, the sample material is positioned over the test fixture in a similar manner to the first test, except that it is repositioned so that 1″ of material extends to the right of the slot. In this way a different area of the sample material is tested than was tested in the first test. The results of these two tests are recorded.

The samples are again returned to the environmental container for a minimum of 15 minutes. For the second set of tests, the samples are removed from the container and placed across the slot opening on the test platform at a 90 degree rotation from the original orientation. This test is performed twice for each material construction and the results are recorded as above.

The reported hand is the average of the four readings recorded.

Thermal Resistance

Thermal Resistance was measured using a testing method based upon ASTM E1530 (“Test Method for Evaluating the Resistance to Thermal Transmission of Thin Specimens of Materials by the Guarded Heat Flow Meter Technique”) using the UNITHERM™ Model 2022 Thermal Conductivity Instrument available from Anter Corporation, Pittsburgh, Pa. A 2 inch diameter test sample of the material is held under a compressive load of 10 psi between two polished metal surfaces each controlled to a different temperature. The upper heater, embedded in the top metal surface, is temperature controlled to 15 degrees C. above the mean sample temperature. The bottom heater is part of a calibrated heat flux transducer, which is attached to a liquid cooled heat sink that is temperature controlled to 15 degrees C. below the mean sample temperature. Guard temperature was controlled to 35 degrees C., while mean sample temperature was controlled to roughly 35 degrees C. An axial temperature gradient is established through the stack as heat flows from the upper surface through the test sample to the heat sink. The temperature drop through the sample is determined from temperature sensors in the metal surfaces on either side of the sample. By measuring this temperature difference (delta Ts) across the test sample and across the heat flux transducer (delta Tr), the thermal resistance can be determined using the ratio of delta Ts to delta Tr. Using a calibration graph of thermal resistance vs. the ratio of delta Ts to delta Tr, that is plotted during the test device calibration, the Thermal Resistance of the test sample can be determined from the calibration curve.

Moisture Vapor Transmission Rate (MVTR)

Samples are die-cut circles of 7.4 cm diameter. The samples are conditioned in a 23° C., 50%±2% RH test room for 4 hours prior to testing.

Test cups are prepared by placing 15 ml of distilled water and 35 g of sodium chloride salt into a 4.5 ounce polypropylene cup, having an inside diameter of 6.5 cm at the mouth. An expanded PTFE membrane (ePTFE), available from W. L. Gore and Associates, Incorporated, Elkton, Md., is heat sealed to the lip of the cup to create a taut, leakproof microporous barrier holding the salt solution in the cup. A similar ePTFE membrane is mounted taut within a 5 inch embroidery hoop and floated upon the surface of a water bath in the test room. Both the water bath and the test room are temperature controlled at 23° C. The sample is laid upon the floating membrane, a salt cup is weighed, inverted and placed upon the sample. After one hour, the salt cup is removed, weighed, and the moisture vapor transmission rate is calculated from the weight pickup of the cup as follows:
MVTR g/(m²×24 hours)=Weight (g) water pickup in cup/[Area (m²) of cup mouth multiplied by the Time (days) of test].

“Moisture vapor permeable” refers to polymer film/textile laminates that have a Moisture Vapor Transmission Rate (MVTR) of at least about 1,000 g/(m²×24 hours).

### EXAMPLES

### Example 1

A rectangular sheet of 2 mil EFEP was positioned above a 10 mil rectangular sheet of a laminate approximately 10 mil thick comprising ePTFE and a brushed polyester fabric. The laminate was of like dimension and positioned such that the edges of the laminate and the EFEP were substantially aligned. Both sheets were folded about a central axis of the long dimension and the edges of the sheets aligned. The long edge opposite the fold and one of the ends was then sealed with a hot iron welder. A refrigeratable gel was poured into the resulting non-laminated pouch between the two layers of EFEP. The open end of the pouch was then heat sealed.

### Example 2

A heat transfer pack with a removable moisture vapor impermeable pouch was constructed. A substantially rectangular sheet of 1 mil EFEP was folded about a central axis of its long dimension and the edges of the sheet were aligned. The long edge and one end were sealed using a hot iron welder. A refrigeratable gel is inserted into the resulting pouch before the moisture vapor impermeable pouch is completely sealed. It may be necessary to provide a release agent if the EFEP sticks to the hot iron welder.

A somewhat larger waterproof, moisture vapor permeable cover was constructed for the impermeable pouch in substantially the same fashion. A slightly larger sheet of 10 mil ePTFE was folded and the long edges and one end were welded using a hot iron welder. The remaining end is left unsealed to allow the impermeable pouch to be inserted.

### Example 3

A heat transfer pack having an inner layer of 1 mil. EFEP and an outer layer consisting of a 10 mil laminate of ePTFE and brushed polyester fabric was assembled in the manner described above in example 1.

### Example 4

A single layer heat transfer pack was assembled from 10 mil ePTFE which was folded and heat sealed in the manner described above in Example 1.

### Test Results

The foregoing examples, as well as other material combinations, were tested for both thermal resistance and fabric hand. These were compared to known cold packs, such as the ACE cold pack, available from Becton Dickinson and Company, Franklin Lakes, N.J. and the 3M Nexcare pack available from 3M Healthcare, St. Paul, Minn. The results reported in Table 1. The conformable heat transfer packs of the present invention are unique in their properties of low fabric hand. Moreover, the novel heat transfer packs do not sacrifice thermal resistance, which is essential to user comfort.

TABLE 1

| | Thermal | | |
|---|---|---|---|
| | Resistance | Handle | |
| | (TR) | (H) | Quotient: |
| Examples | (deg C. in² /W) | (g) | (TR/H) |
| ACE Cold Pack | 6.9 | 113 | 0.061 |
| 3M Nexcare | 9.6 | 198 | 0.049 |
| Example 1 | 6.9 | 35 | 0.196 |
| Example 2 | 7.3 | 72 | 0.101 |
| Example 3 | 6.1 | 67 | 0.091 |
| Example 4 | 5.7 | 82 | 0.070 |

While particular embodiments of the present invention have been illustrated and described herein, the present invention should not be limited to such illustrations and descriptions. It should be apparent that changes and modifications may be incorporated and embodied as part of the present invention within the scope of the following claims.

1. A heat transfer pack comprising:
a) a heat transfer material; and
b) a pouch surrounding the heat transfer material, the pouch comprising a moisture vapor impermeable inner layer and a waterproof, moisture vapor permeable outer layer.

2. The heat transfer pack of claim 1 in which outer layer comprises ePTFE.

3. The heat transfer pack of claim 1 in which the outer layer comprises porous polyethylene.

4. The heat transfer pack of claim 1 in which the outer layer is a laminate comprising a porous membrane and at least one textile layer.

5. The heat transfer pack of claim 4 in which the textile layer comprises a polyester fabric.

6. The heat transfer pack of claim 1 in which the outer layer comprises a porous membrane and a filler.

7. The heat transfer pack of claim 6 in which the filler is selected from the group of: pigments, colorants, scents, medicants, anti-microbials, antibiotics, antibacterial agents, antifungals, dentifrice, remineralizing agents, immunological agents, anti-inflammatory agents, hemostatic agents, analgesics and mixtures thereof.

8. The heat transfer pack of claim 1 in which the outer layer is oleophobic.

9. The heat transfer pack of claim 1 in which the outer layer is hydrophobic.

10. The heat transfer pack of claim 1 in which the pouch comprises a conformable insulating layer, such that the quotient of the thermal resistance divided by the fabric hand of the pouch is greater than about 0.05, and the pouch having a fabric hand value of less than about 100 g.

11. The heat transfer pack of claim 1 in which the pouch comprises a conformable insulating layer, such that the quotient of the thermal resistance divided by the fabric hand of the pouch is greater than about 0.05, and the pouch having a fabric hand value of less than about 90 g.

12. The heat transfer pack of claim 1 in which the pouch comprises a conformable insulating layer, such that the quotient of the thermal resistance divided by the fabric hand of the pouch is greater than about 0.05, and the pouch having a fabric hand value of less than about75 g.

13. The heat transfer pack of claim 1 in which the pouch comprises a conformable insulating layer, such that the quotient of the thermal resistance divided by the fabric hand of the pouch is greater than about 0.05, and the pouch having a fabric hand value of less than about 50 g.

14. The heat transfer pack of claim 1 in which the pouch comprises a conformable insulating layer, such that the quotient of the thermal resistance divided by the fabric hand of the pouch is greater than about 0.05, and the pouch having a fabric hand value of less than about 30 g.

15. The heat transfer pack of claim 1 in which the outer layer substantially surrounds the inner layer.

16. The heat transfer pack of claim 1 in which the outer layer envelops the inner layer.

17. The heat transfer pack of claim 1 in which the outer layer is removable from the inner layer.

18. The heat transfer pack of claim 1 in which the inner layer and the outer layer are bonded together near the periphery of the pouch.

19. The heat transfer pack of claim 1 in which the inner layer comprises a thermoplastic.

20. The heat transfer pack of claim 4 in which the inner layer comprises a thermoplastic selected from the group of EFEP, FEP, PFA, THV, PVDH, PU and PE.

21. An article for containing heat transfer material, the article comprising a nonlaminated composite pouch, the pouch comprising a waterproof, moisture vapor permeable outer layer substantially surrounding a moisture vapor impermeable inner layer.

22. The article of claim 21 in which the waterproof, moisture vapor permeable outer layer comprises ePTFE.

23. The article of claim 21 in which the waterproof, moisture vapor permeable outer layer comprises porous polyethylene.

24. The article of claim 21 in which the waterproof, moisture vapor permeable cover is a laminate comprising a porous membrane and at least one textile layer.

25. The article of claim 24 in which the textile layer comprises a polyester fabric.

26. The article of claim 21 in which the waterproof, moisture vapor permeable cover comprises a porous membrane and a filler.

27. The article of claim 26 in which the filler is selected from the group of: pigments, colorants, scents, medicants, anti-microbials, antibiotics, antibacterial agents, antifungals, dentifrice, remineralizing agents, immunological agents, anti-inflammatory agents, hemostatic agents, analgesics and mixtures thereof.

28. The article of claim 21 in which the waterproof, moisture vapor permeable outer layer is oleophobic.

29. The article of claim 21 in which the waterproof, moisture vapor permeable outer layer is hydrophobic.

30. The article of claim 21 in which the moisture vapor impermeable inner layer and the waterproof, moisture vapor permeable outer layer are bonded together near the periphery of the article.

31. The article of claim 21 in which the pouch comprises a conformable insulating layer, such that the quotient of the thermal resistance divided by the fabric hand of the pouch is greater than about 0.05, and the pouch having a fabric hand value of less than about 100 g.

32. The article of claim 21 in which the pouch comprises a conformable insulating layer, such that the quotient of the thermal resistance divided by the fabric hand of the pouch is greater than about 0.05, and the pouch having a fabric hand value of less than about 90 g.

33. The article of claim 21 in which the pouch comprises a conformable insulating layer, such that the quotient of the thermal resistance divided by the fabric hand of the pouch is greater than about 0.05, and the pouch having a fabric hand value of less than about 75 g.

34. The article of claim 21 in which the pouch comprises a conformable insulating layer, such that the quotient of the thermal resistance divided by the fabric hand of the pouch is greater than about 0.05, and the pouch having a fabric hand value of less than about 50 g.

35. The article of claim 21 in which the pouch comprises a conformable insulating layer, such that the quotient of the thermal resistance divided by the fabric hand of the pouch is greater than about 0.05, and the pouch having a fabric hand value of less than about 30 g.

36. The heat transfer pack of claim 21 in which the inner layer comprises a thermoplastic.

37. The heat transfer pack of claim 21 in which the inner layer comprises a thermoplastic selected from the group of EFEP, FEP, PFA, THV, PVDH, PU and PE.

38. An article for containing heat transfer material comprising a waterproof, moisture vapor permeable pouch having an inner surface and an outer surface, the pouch having an opening therein of sufficient size to permit insertion and removal of heat transfer material.

39. The article of claim 38 wherein the waterproof, moisture vapor permeable pouch comprises ePTFE.

40. The article of claim 38 wherein the waterproof, moisture vapor permeable pouch comprises porous polyethylene.

41. The article of claim 38 in which the waterproof, moisture vapor permeable pouch is a laminate comprising a porous membrane and at least one textile layer.

42. The article of claim 41 in which the textile layer comprises a polyester fabric.

43. The article of claim 38 in which the pouch comprises a porous membrane and a filler.

44. The article of claim 43 in which the filler is selected from the group of: pigments, colorants, scents, medicants, anti-microbials, antibiotics, antibacterial agents, antifungals, dentifrice, remineralizing agents, immunological agents, anti-inflammatory agents, hemostatic agents, analgesics and mixtures thereof.

45. The article of claim 38 in which the outer surface of the waterproof, moisture vapor permeable pouch comprises an ePTFE membrane.

46. The article of claim 45 in which the ePTFE membrane is hydrophobic.

47. The article of claim 45 in which the ePTFE membrane is oleophobic.

48. The article of claim 38 in which the pouch comprises a conformable insulating layer, such that the quotient of the thermal resistance divided by the fabric hand of the pouch is greater than about 0.05,. and the pouch having a fabric hand value of less than about 100 g.

49. The article of claim 38 in which the pouch comprises a conformable insulating layer, such that the quotient of the thermal resistance divided by the fabric hand of the pouch is greater than about 0.05, and the pouch having a fabric hand value of less than about 90 g.

50. The article of claim 38 in which the pouch comprises a conformable insulating layer, such that the quotient of the thermal resistance divided by the fabric hand of the pouch is greater than about 0.05, and the pouch having a fabric hand value of less than about 75 g.

51. The article of claim 38 in which the pouch comprises a conformable insulating layer, such that the quotient of the thermal resistance divided by the fabric hand of the pouch is greater than about 0.05, and the pouch having a fabric hand value of less than about 50 g.

52. The article of claim 38 in which the pouch comprises a conformable insulating layer, such that the quotient of the thermal resistance divided by the fabric hand of the pouch is greater than about 0.05, and the pouch having a fabric hand value of less than about 30 g.

53. The article of claim 38 further comprising a closure disposed at the opening.

54. The article of claim 53 wherein the closure is moisture impermeable.

55. A heat transfer pack for therapeutic application to a patient comprising:
a) a heat transfer material;
b) a moisture vapor impermeable pouch enclosing the heat transfer material, the pouch having an exterior surface; and
c) a waterproof, moisture vapor permeable patient contacting material attached to the exterior surface.

56. The heat transfer pack of claim 55 in which the patient contacting material comprises ePTFE.

57. The heat transfer pack of claim 55 in which the patient contacting material comprises porous polyethylene.

58. The heat transfer pack of claim 55 in which the patient contacting material is a laminate comprising a porous membrane layer and at least one textile layer.

59. The heat transfer pack of claim 58, in which the textile layer comprises a polyester fabric.

60. The heat transfer pack of claim 55, wherein the patient contacting material is oleophobic.

61. The heat transfer pack of claim 55, wherein the patient contacting material is hydrophobic.

62. A heat transfer pack comprising:
a) a heat transfer material; and
b) a pouch surrounding the heat transfer material, the pouch comprising a moisture vapor impermeable inner layer and a waterproof, moisture vapor permeable outer layer, said outer layer comprising a laminate, said laminate comprising ePTFE and a polyester fabric.

63. The heat transfer pack of claim 62 in which the heat transfer material is a gel.

64. The heat transfer pack of claim 62 in which the inner layer comprises EFEP.

65. The heat transfer pack of claim 62 in which the inner layer comprises polyurethane.

66. The heat transfer pack of claim 62 in which the pouch comprises a conformable insulating layer, such that the quotient of the thermal resistance divided by the fabric hand of the pouch is greater than about 0.05, and the pouch having a fabric hand value of less than about 75 g.

67. The article of claim 62 in which the pouch comprises a conformable insulating layer, such that the quotient of the thermal resistance divided by the fabric hand of the pouch is greater than about 0.05, and the pouch having a fabric hand value of less than about 30 g.

68. A heat transfer pack comprising:
a) a heat transfer material;
b) a multi-layer pouch surrounding the heat transfer material, said multi-layer pouch comprising a moisture vapor impermeable inner pouch; and a waterproof, moisture vapor permeable outer pouch substantially surrounding the inner pouch, said outer pouch comprising a laminate, said laminate comprising ePTFE and a polyester fabric.

69. The heat transfer pack of claim 66 in which the heat transfer material is a gel.

70. The heat transfer pack of claim 66 in which the inner pouch comprises EFEP.

71. The heat transfer pack of claim 66 in which the inner pouch comprises polyurethane.

72. The heat transfer pack of claim 66 in which the quotient of the thermal resistance divided by the fabric hand of the multi-layer pouch is greater than about 0.05, and the multi-layer pouch having a fabric hand value of less than about 75 g.

73. The heat transfer pack of claim 66 in which the quotient of the thermal resistance divided by the fabric hand of the multi-layer pouch is greater than about 0.05, and the multi-layer pouch having a fabric hand value of less than about 30 g.
